(19) United States
(12) Patent Application Publication
(10) Pub. No.: US 2012/0152847 A1
(43) Pub. Date: Jun. 21, 2012

(54) SORBENT FOR ENDOTOXINS

(76) Inventors: Dieter Falkenhagen, Krems (AT); Viktoria Weber, Krems (AT)

(21) Appl. No.: 13/146,190

(22) PCT Filed: Jan. 20, 2010
(86) PCT No.: PCT/AT10/00017

§ 371 Date: Mar. 12, 2012

(30) Foreign Application Priority Data
Jan. 22, 2009 (AT) A 111/2009

(51) Int. Cl. A61M 1/36; B01D 15/08; B01J 20/22

(52) U.S. Cl. 210/691; 502/1; 210/263

(57) ABSTRACT

The invention relates to a sorption agent for removing endotoxins from a biological fluid, the sorption agent having a water-insoluble, porous carrier and polymyxin, which is immobilized on the carrier, the carrier having a neutral, hydrophobic surface and polymyxin being immobilized on the surface of the carrier via hydrophobic interaction. The sorption agent is used in extracorporeal blood purification, in particular for treating individuals with sepsis.

The invention relates to a sorption agent for removing endotoxins from a biological fluid, the sorption agent having a water-insoluble, porous carrier and polymyxin, which is immobilized on the carrier.

Endotoxins are lipopolysaccharides (LPS) in the cell wall of gram-negative bacteria and are released by cell lysis. In fact, lipopolysaccharides are the most frequent lipid component of the external cell membrane of gram-negative bacteria. Endotoxins are pyrogenic substances, i.e., the affected individual reacts with a strong inflammation reaction and fever if endotoxins reach the body, for example, in the course of microbial poisoning, and display systemic effect. The presence of endotoxins in the blood circuit results in an uncontrolled activation of the immune cells and an imbalance of the coagulation system. Depending on their concentration, they may result in sepsis, which is characterized, inter alia, by high fever, low blood pressure, and, in severe cases, by multi-organ failure. Sepsis is a disease to be taken very seriously; the lethality of individuals having severe sepsis or septic shock is approximately 30-60%, depending on the degree of severity of the disease. Patients having impaired immune defense, such as liver patients or patients in chemotherapy, also tend toward bacterial infections and thus display symptoms of endotoxin poisoning.

The structure of a lipopolysaccharide molecule is in three parts: A lipid A forms the area of the molecule which faces toward the bacteria cell; the molecule is anchored in the external membrane of the gram-negative bacteria by the lipid A. The LPS molecule further has a middle, highly conserved core region, which is bound to the lipid A. The third and outermost area is formed by an 0-specific polysaccharide (0-antigen), whose structure can vary strongly within the various gram-negative bacteria. The toxic effect is to be attributed to the lipid A, which is first released during the cell lysis.

Endotoxins can be removed from a biological fluid, such as blood or plasma, which is contaminated by endotoxins, using a suitable sorption agent. The treatment of patients having endotoxin poisoning or sepsis is performed in particular in the scope of extracorporeal blood purification (apheresis).

Apheresis methods are extracorporeally performed methods, in which pathophysiologically-relevant blood and plasma components, for example, biomolecules such as (glyco)proteins, peptides, lipids, lipoproteins, and lipopolysaccharides, but also blood cells and blood plasma, are removed. Apheresis methods can be used for diagnostic and therapeutic purposes, on the one hand, they also represent a very effective possibility for obtaining specific blood components from healthy individuals in a sufficient quantity and in sufficiently high purity, on the other hand. Great significance is ascribed to the therapeutic apheresis, since for specific indications, this is often a very effective alternative, which simultaneously has few side effects, to medicinal treatment. Thus, in plasmapheresis methods, the plasma can either be completely separated and replaced by a substitution solution, or only specific components such as LDL, endotoxins, or immunoglobulins are removed therefrom using a sorption agent and the plasma is subsequently returned to the donor/patient.

To remove endotoxins from a biological fluid (typically blood or blood plasma) which is contaminated with endotoxins, it is brought into close contact with a sorption agent, which is typically located in a sorption apparatus. The endotoxins are bound to the surface of the sorption agent and are removed from the biological fluid. The biological fluid which is freed from endotoxins is returned to the patient. The sorption apparatus is either situated on the blood side in an extracorporeal blood circuit or on the filtrate side in a plasma circuit of an extracorporeal blood purification device. The endotoxin binding capacity and speed are a function of the composition of the sorption agent.

The speed of the endotoxin binding by the sorption agent is decisive for the survival of the patient. The time which remains to remove the endotoxins from the blood of a patient is very short (<12 hours) and can be only a few hours in the case of severe sepsis.

It has been shown that anion exchange resins (e.g., DEAE or PEI groups bound to cellulose) are very well suitable for endotoxin binding. However, the undesired binding of important factors of the intracorporeal coagulation system such as protein C and protein S and the coagulation problems connected thereto are disadvantageous.

These coagulation problems can be avoided by the use of a specific sorption agent, which has immobilized antibodies against endotoxins. However, this possibility only has limited applicability for economic reasons.

Sorption agents of the type mentioned at the beginning, in which polymyxin molecules are immobilized on a water-insoluble, porous carrier, have become known as very useful alternatives.

Polymyxins are antibiotic substances for treating infections with gram-negative bacteria. Polymyxins engage in the cell wall structure, in that they increase the permeability of the cell membrane, because of which cell lysis occurs. Polymyxins bind not only phospholipids, but rather also lipopolysaccharides (endotoxins) with high affinity. Because of the neurotoxic and nephrotoxic effect of the polymyxins, only polymyxin B and polymyxin E (colistin) have received a certain therapeutic significance. Polymyxin B and/or polymyxin E are therefore only applicable for oral and topical treatment forms. They are unsuitable for parenteral, systemic treatment of endotoxin poisoning or sepsis because of their toxic effect.

However, it has proven to be favorable to covalently bind polymyxin, in particular polymyxin B, to a water-insoluble carrier and to use the polymyxin B-coated carrier as the sorption agent for removing endotoxins from contaminated biological fluids.

Polymyxin B-immobilized carriers made of porous glass (FPG 2000) and polymyxin B-immobilized polysaccharide carriers based on cellulose (Cellulofine A-3) are disclosed in EP 0110 409 A. Microparticles made of cellulose or derivatized cellulose, to which polymyxin B is covalently bonded, are also known. [Weber V., Loth F., Linsberger I., Falkenhagen D.: Int. J. Artif. Organs 25(7), 679] A high level of endotoxin adsorption can be achieved using polymyxin-coated cellulose carriers, however, they have the disadvantage that polymyxin is covalently bonded to these carriers and the cellulose must therefore be chemically activated before the binding of the polymyxin.

EP 0 129 786 A2 describes an endotoxin detoxification material having a fibrous carrier, on which polymyxin is covalently immobilized. The fibrous carrier is equipped with functional groups to covalently bond polymyxin to the surface of the carrier. The endotoxin detoxification material from EP 0129 786 is on the market as a filler material for an adsorption module (trade name: Toraymyxin) [Shoji H. 2003. Extracorporeal endotoxin removal for the treatment of sepsis: endotoxin adsorption cartridge (Toraymyxin)] and at the moment it is the only sorption agent which is authorized for clinical treatment of sepsis in the scope of extracorporeal blood purification. A critical review of the effectiveness of fibers carriers having immobilized polymyxin B, in which the quality of the treatment is represented as suboptimal, has only recently been published [Cruz D N et al. 2007; Effectiveness of polymyxin B-immobilized fiber column in sepsis: a systematic review. Crit. Care 11(3):137].

The known sorption agents which are based on binding of the endotoxins by polymyxin have the disadvantage of low endotoxin binding capacity and speed. Since polymyxin is bound to the polymer via NH₂ groups, the access for endotoxins is impaired. Further disadvantages result from the costly and complex production method and the higher production costs connected thereto.

Although the lethality of patients having endotoxin toxicity, in particular sepsis, has been able to be reduced by the clinical application of the above-mentioned Toraymyxin adsorption module, the lethality of patients having severe sepsis and septic shock is still very high in spite of maximum therapy. For this reason and because of the high and rising incidence of septic states, there is a high demand for a sorption agent having improved sorption performance for endotoxins.

It is an object of the invention to provide a sorption agent, using which endotoxins can be removed to a high degree and at high speed from a biological fluid.

The object is achieved by a sorption agent of the type mentioned at the beginning, in which according to the invention the carrier has a neutral, hydrophobic surface and polymyxin is immobilized via hydrophobic interaction on the surface of the carrier.

Thanks to the sorption agent according to the invention, a significant improvement of the endotoxin sorption capacity and endotoxin sorption speed has been able to be achieved. The inventors have established to an unexpected extent that—in comparison to the known sorption agents—a large endotoxin quantity can already be bound from an endotoxin-contaminated biological fluid after a short action time by the sorption agent according to the invention. This has great therapeutic advantages, in particular for patients having sepsis, since a large volume of biological fluid can be freed of endotoxins in a short time. The survival chances of patients having severe sepsis can be improved by the sorption agent according to the invention. As already noted, the speed of the endotoxin binding by the sorption agent is decisive for the survival of the patient. The treatment duration in the scope of extracorporeal blood purification can also be shortened thanks to the sorption agent according to the invention, whereby chronological, financial, and human resources can be saved.

A further advantage of the invention in relation to the known sorption agents is its particularly simple production, since polymyxin is immobilized (using the hydrophobic section of the polymyxin molecule) via hydrophobic interaction on the neutral, hydrophobic surface of the carrier. The increase of the endotoxin binding effectiveness of the sorption agent according to the invention may be explained in that the binding mediated using hydrophobic interaction leaves the NH₂ groups of the polymyxin exposed and these are available essentially in their entirety for the endotoxin binding. No complex chemical steps are required for immobilizing the polymyxin on the carrier. The production of the sorption agent according to the invention can therefore be performed economically and reproducibly.

The hydrophobic interaction has great biochemical significance and is based on the phenomenon that hydrophobic molecules tend toward association in a polar environment. The hydrophobic interaction is therefore not a force per se, but rather is compelled by a polar environment. In the present invention, the hydrophobic interaction occurs between the hydrophobic section of the polymyxin molecule and the neutral, hydrophobic internal and external surfaces of the porous carrier.

The term “sorption agent” in the context of this disclosure is to be understood as an agent for performing a sorption, preferably an adsorption, i.e., molecules which are located in a biological fluid are fixed by the surface forces of the sorption agent. In the description, the terms “adsorption agent” or “adsorbent” or “adsorber” are also used instead of the term “sorption agent”. According to the invention, the sorption agent is provided for the adsorption of endotoxins from a biological fluid contaminated with endotoxins. The sorption agent according to the invention is used above all in extracorporeal blood purification, in particular in patients having septic states.

The expression “biological fluid” used in the scope of the invention can relate to cell-free liquids, in particular blood plasma, or to liquids containing cells, in particular blood. Since it is also necessary in the course of extracorporeal blood purification to introduce other liquids, for example, solutions containing coagulation inhibitors (heparin solution, citrate solution) or substitution solutions (electrolytes, liquids to compensate for the liquid loss) into the extracorporeal blood circuit or into a blood plasma circuit, a biological fluid is also to be understood as diluted blood or diluted blood plasma. The invention is primarily intended for the field of human medicine and therefore primarily relates to human biological fluids. However, this does not preclude the invention also being used in the field of veterinary medicine.

Polymyxins are known chemical compounds which originally originate from the bacteria Bacillus polymyxa. Polymyxin B and polymyxin E (Colistin) are to be noted in particular.

The term “water-insoluble, porous carrier, which has a neutral, hydrophobic surface” as used in independent Claim 1 relates in the context of this disclosure to a porous, water-insoluble solid, which has external and internal surfaces. The external and internal surfaces are neutral and hydrophobic. The term “neutral” is to be understood as non-ionic. The invention is directed above all to particulate carriers.

It is particularly expedient in practice if the carrier is a hydrophobic polymer. Good reproducibility of the carrier material can thus be ensured, in particular with respect to the porosity and the particle size. The porosity and the particle size may additionally be varied very well. The hydrophobic polymer can be both a homopolymer and also a heteropolymer. Cross-linked polystyrene polymers have proven to be particularly favorable for practical performance. During extracorporeal blood purification, there are high requirements for the sterility of the device components which come into contact with the bodily fluids of the patient. This also applies to sorption agents. Cross-linked polystyrene polymers are distinguished by high stability with respect to heat and chemicals and are already established in clinical practice.

The strength of the hydrophobic interaction between polymyxin and carrier is determined, on the one hand, by the hydrophobicity of the neutral, hydrophobic carrier and, on the other hand, by the ionic strength of the medium. As already mentioned above, polymyxin has neurotoxic and nephrotoxic effects. Therefore, the most solid possible binding of the polymyxin to the external and internal surfaces of the carrier is desired. In a particularly preferred variant, the cross-linked polystyrene polymer is a polystyrene-divinyl benzene copolymer. The surface of a polystyrene-divinyl benzene copolymer has a high hydrophobicity, whereby very strong binding of the polymyxin to the carrier surface is achieved. The inventors have established that after the immobilization of the polymyxin via hydrophobic interaction, no polymyxin is released into the biological fluid. However, it is also possible to use other neutral, hydrophobic polymers of high hydrophobicity, which are well known to a person of skill in the relevant art. The inventors have been able to establish that no desorption of the polymyxin and therefore also no losses of the endotoxin binding are measurable upon autoclaving of the sorption agent according to the invention. This is very advantageous for the patient safety.

Furthermore, it has been found that the pore size of the porous carrier is also significant with respect to the endotoxin adsorption. It is therefore favorable, also for reasons of reproducibility, if the porous carrier has a defined mean pore size. The mean pore size of the carrier always relates to that before the mobilization of the polymyxin via hydrophobic interaction.

The mean pore size can be set particularly well if the porous carrier is produced from a synthetic polymer. Although a person skilled in the art in this field knows what the term “mean pore size of a polymer” is to be understood as and how the porosity or the mean pore size can be intentionally set, this term will nonetheless be briefly defined here for reasons of clarity. The mean pore size relates to the mean diameter of the pores. In a Gaussian size distribution of the pore diameters, the mean pore diameter is the pore diameter which corresponds to the maximum of the distribution curve. The mean pore diameter can be determined using nitrogen adsorption (as described in Weber et al. 2008. Neutral styrene divinylbenzene copolymers for adsorption of toxins in liver failure. Biomacromolecules 9(41322-1328) or using mercury intrusion, for example. The pore size of a polymer is set by variation of the concentration of the participating monomers or co-monomers, the solvent, or the modulator. The smaller the pores of the polymer are selected to be, the larger the internal surface area of the polymer which is available for sorption, in particular adsorption. The larger the pores, the better the accessibility of the pores for larger molecules. A production method for a synthetic, hydrophobic polymer of defined pore size, as can be used for the invention, is described in the above-mentioned publication by Weber et al.

It has been shown that particularly good endotoxin sorption can be achieved by the sorption agent if the carrier has a mean pore size of at least 15 nm. The carrier preferably has a mean pore size of at least 30 nm. For clinical application of extracorporeal blood purification, however, it is favorable if the mean pore size of the uncoated carrier is not greater than 120 nm. The internal surface area of the sorption agent would otherwise become too small; the result would be a reduction of the endotoxin sorption capacity (endotoxin adsorption capacity). In laboratory experiments (see examples below), very good results were obtained when the uncoated carrier had a mean pore size of 30-40 nm.

In a particularly advantageous variant, the uncoated carrier has a mean pore size of approximately 80-100 nm. In this variant, the elimination of endotoxins from a biological fluid occurs with particularly high speed and high efficiency. Only a small quantity of sorption agent is therefore required to bind a large quantity of endotoxin. For example, the concentration of this variant of the sorption agent according to the invention, when it is used as a suspension in an extracorporeal plasma circuit, can be selected as 1% (weight-percent volume-percent). An extracorporeal plasma circuit which contains a suspension of a sorption agent in the form of microparticles represents a central component of a Microspheres-based Detoxification System (MDS). An MDS is known from EP 0776223 B and U.S. Pat. No. 5,855,782.

In addition, the form of the sorption agent during the sorption procedure is also significant. In an advantageous variant, the sorption agent according to the invention is in the form of microparticles. The particle size influences the kinetics of the adsorption. In addition, with a small particle size, there is a large surface area/volume ratio. In an advantageous subvariant, the microparticles have a particle size of 20 μm or less.

The microparticles are used in particular in an MDS, which was already mentioned above. The microparticles circulate as a suspension in a purification circuit (plasma circuit) on the filtrate side of a membrane filler. However, if the membrane filler leaks, the danger exists that microparticles will reach the extracorporeal blood circuit and then the body of the patient and will result in a lung embolism therein. For this reason, it is advantageous in a further subvariant if the microparticles have a particle size of 8 μm or less, ideally 5 μm or less, since the danger of a lung embolism can be avoided at these small particle sizes.

The sorption agent according to the invention is primarily provided for use in extracorporeal blood purification (apheresis).

In an important application of the invention, the sorption agent can be used as a filler material for a sorption apparatus. The sorption apparatus can be implemented as a column or cartridge. Depending on which blood purification device or which blood purification method (hemoperfusion, plasmapheresis/plasmasorption) is used, the sorption apparatus can be situated on the blood side in an extracorporeal blood circuit or in a plasma circuit on the filtrate side. The biological fluid (blood or blood plasma) passes the sorption apparatus, the endotoxins binding to the immobilized polymyxin molecules of the sorption agent. The purified blood or plasma is returned to the patient.

A further possible use relates to a plasma circuit, in which the sorption agent is provided distributed as a suspension in the plasma. An example of such a plasma circuit is found as a device element in an above-described MDS. The sorption agent provided in suspension in a plasma circuit is preferably in the form of microparticles.

Although the endotoxin sorption agent according to the invention is primarily provided for use in extracorporeal blood purification (apheresis), usage in chromatography is also conceivable. The sorption agent can thus be used as a filler material for chromatography columns for purifying endotoxin-loaded blood or blood plasma. Other applications for removing endotoxins from biological fluids or water are also conceivable.

The sorption agent according to the invention or a sorption apparatus containing a sorption agent according to the invention or a plasma circuit containing a suspension of a sorption agent according to the invention is particularly suitable for treating a sepsis.

Furthermore, the invention relates to a method for removing endotoxins from a biological fluid, in which a biological fluid contaminated with endotoxins is brought into contact with the sorption agent according to the invention. As described above, the biological fluid can pass a sorption apparatus which contains the sorption agent. However, the sorption agent can also be suspended in the biological fluid. An example of the latter is the above-described MDS. The biological fluid can be blood or blood plasma.

The present invention is explained in greater detail hereafter on the basis of nonrestrictive examples.

### 1. EXAMPLE 1

### Production of Sorption Agents According to the Invention (Adsorbers)

To produce sorption agents according to the invention, neutral, hydrophobic polystyrene-divinyl benzene copolymers of various pore sizes were coated using polymyxin B, i.e., polymyxin B was adsorbed via hydrophobic interactions on the external and internal surfaces of the polystyrene-divinyl benzene copolymers.

### 1.1. Providing Neutral, Hydrophobic Polymers:

Polystyrene-divinyl benzene copolymers (referred to in short as “polymers” or “carriers” or “uncoated adsorbers”) of various mean pore sizes are listed in Table 1.

TABLE 1
Polystyrene-divinyl benzene copolymers

| | Mean pore size |
|---|---|
| designation | [nm] |
| #1822 | 15-20 |
| #1823 | 15-20 |
| #1824 | 30-40 |
| #1825 | 80-100 |
| #1826 | 80-100 |

The particle size of the polymers was 5 μm+/−3-4 μm.

### 1.2. Polymyxin Coating of the Polystyrene-Divinyl Benzene Copolymers:

To produce the adsorbers according to the invention, the polymers listed in Table 1 were coated using polymyxin B.

Polymyxin B (PMB) was obtained from Sigma (catalog number: 81334, lot: 1348744). For the coating, a polymyxin B solution (PMB solution) was produced, 50 mg PMB being dissolved in 10 mL LAL water.

Each 1 g of polymer damp weight was admixed with 5 mL PMB solution and incubated for 60 minutes in an Enviro Genie® Shaker at room temperature.

The mixture was then centrifuged at 4000 g for 15 minutes, the supernatant was withdrawn, and the sediment was admixed with 10 mL 0.9% NaCl and vortexed. This step was repeated 3 to 5 times. Subsequently, the mixture was once again centrifuged at 4000 g for 15 minutes, the supernatant was withdrawn, and a 50% adsorber suspension was produced in pyrogen-free 0.9% NaCl. The polymyxin B-coated adsorbers are listed in Table 2.

TABLE 2
Polymyxin B-coated adsorbers

| Designation of the polymyxin | Mean pore size [nm] of |
|---|---|
| B-coated adsorbers | the uncoated adsorber |
| #1822 + PMB | 15-20 |
| #1823 + PMB | 15-20 |
| #1824 + PMB | 30-40 |
| #1825 + PMB | 80-100 |
| #1826 + PMB | 80-100 |

### 2. EXAMPLE 2

### Endotoxin Adsorption—Batch Test

Polymyxin B-coated adsorbers #1825+PMB and #1826+PMB having a mean pore size (PS) of the polymers of 80-100 nm (see first Example 1—Table 2) were compared to corresponding uncoated adsorbers #1825 and #1826 with respect to the endotoxin binding.

2.1. Adsorbers and Adsorber Preparation:

The following adsorbers (PS=80-100) were prepared according to the protocol from Example 1:

1) #1825

2) #1826

3) #1825+PMB (PMB-coated)

4) #1826+PMB (PMB-coated)

The polymyxin B-coated adsorbers and the uncoated adsorbers were washed—as described in 1.2-5 times using pyrogen-free NaCl. A 50% adsorber suspension was finally produced in pyrogen-free NaCl.

### 2.2. Heparin Plasma:

25 ml heparin plasma was acquired from a donor (5×9 ml whole blood withdrawal).

### 2.3. Endotoxin Solution:

LPS Pseudomonas aeruginosa (Sigma, L7018, batch 109H4043). The portioned (each 100 μl 10 ⁻³ g/ml (1 mg/ml)) endotoxins, which were stored at −70° C. in microcentrifuge tubes, were thawed and admixed with 900 μl LAL water. The endotoxins were subsequently further diluted in NaCl to the required concentration of 50 ng/ml (=endotoxin solution; ET solution).

2.4. Test Batches:

In the batch of the test, a further dilution of the endotoxin solution was performed to a final concentration of 5 ng/ml. The adsorber final concentration was 10%. For the test batches, 50% adsorber suspension (vol. ads. suspension), heparin plasma (plasma), and the endotoxin solution [50 ng/ml] (ET solution) were pipetted into test tubes according to the specifications of Table 3. As controls, a test tube without adsorber (control) and a test tube having untreated heparin plasma (plasma) were carried along.

TABLE 3
Test batches

| Adsorber | Vol. Ads. Suspension | | Plasma | ET solution |
|---|---|---|---|---|
| #1825 | 600 | μL | 2100 μL | 300 μL |
| #1826 | 600 | μL | 2100 μL | 300 μL |
| #1825 + PMB | 600 | μL | 2100 μL | 300 μL |
| #1826 + PMB | 600 | μL | 2100 μL | 300 μL |
| Control | 100 | μL NaCl | 800 μL | 100 μL |

| | | | |
|---|---|---|---|
| Plasma | 0 | 100 μL | 0 |

The test batches were incubated at 37° C. for 60 minutes in the Enviro-Genie shaker.

### 2.5. Sampling:

All steps of the batch test and the LAL test were performed in glass LAL test tubes (T100, with plastic cap) from Chromogenix (also dilutions and standard curves). Only the sampling for centrifuging was performed in pyrogen-free microcentrifuge tubes.

After 5, 15, and 60 minutes incubation time, the tubes were vortexed. 0.2 mL sample was taken in each case and transferred into pyrogen-free microcentrifuge tubes. The samples were centrifuged in a centrifuge (Eppifuge) for 10 minutes at 11,000 g. 100 μL supernatant was transferred into glass LAL test tubes and tested in the LAL test.

| 2.6. Materials: | | Batch: |
|---|---|---|
| microtitration plates MT 1007 | CoaChrom | 02430103 |
| LAL test Tubes T 200 | Ch. River Endosafe | 53351 D |
| Combitips plus 2.5 Biopur | Eppendorf | V126339 |
| Pipette tips | Eppendorf | V125542M |
| Pipette tips | Eppendorf | W130324Q |
| NaCl 0.9% | Mayerhofer | |
| Microcentrifuge tubes | Greiner | 05200108 |
| Charles River Endosafe | | |
| Endochrome | kit. lot: | W3322CTK6 |

### 2.7. Results:

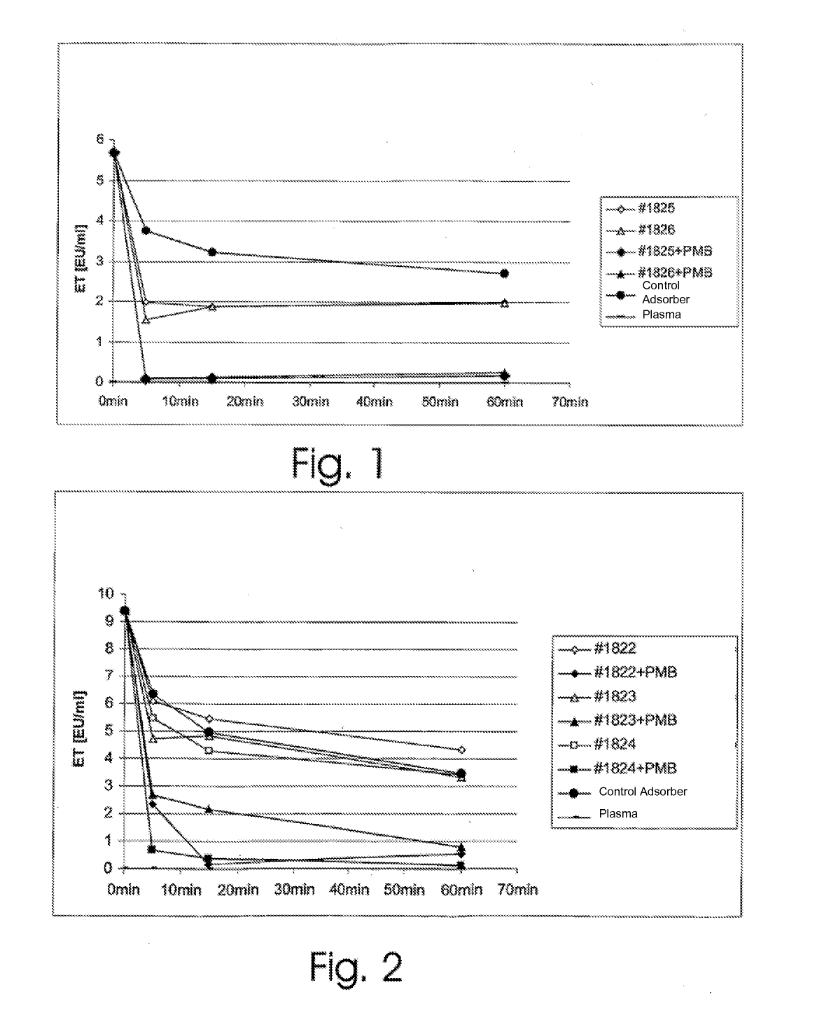

The results are listed in Table 4 and shown in FIG. 1, the curves shown in FIG. 1 representing the endotoxin (ET) adsorption (in endotoxin units (EUI/ml) of PMB-coated adsorbers (mean pore size 80-100 nm) in the time curve.

TABLE 4
Results of batch test - endotoxin adsorption

| EU/ml | 0 min | 5 min | 15 min | 60 min |
|---|---|---|---|---|
| # 1825 | 5.662 | 1.98 | 1.88 | 2.00 |
| # 1826 | 5.662 | 1.539 | 1.88 | 1.98 |
| # 1825 + PMB | 5.662 | 0.0665 | 0.09 | 0.17 |
| # 1826 + PMB | 5.662 | 0.09 | 0.13 | 0.26 |
| Control without ads. | 5.662 | 3.749 | 3.22 | 2.70 |
| Native plasma | 0 | 0 | 0 | 0 |

### 3. EXAMPLE 3

### Endotoxin Adsorption—Batch Test

Polymyxin B-coated adsorbers #1822+PMB, #1823+PMB, and #1824+PMB having a mean pore size (PS) of 15-20 nm or 30-40 nm were compared to corresponding uncoated adsorbers #1822, #1823, and #1824 with respect to the endotoxin binding.

### 3.1. Adsorbers and Adsorber Preparation:

The following adsorbers were prepared according to the protocol of Example 1:

1) #1822

2) #1823

2) #1824

3) #1822+PMB (PMB-coated)

4) #1823+PMB (PMB-coated)

5) #1824+PMB (PMB-coated)

The polymyxin B-coated adsorbers and the uncoated adsorbers were washed—as described in 1.2—5 times using pyrogen-free NaCl. A 50% adsorber suspension was finally produced in pyrogen-free NaCl.

3.2. Heparin plasma and endotoxin solution: corresponding to Sections 2.2 and 2.3.

### 3.3. Test Batches:

In the batch of the test, a further dilution of the endotoxin solution was performed to a final concentration of 5 ng/ml. The adsorber final concentration was 10%. For the test batches, 50% adsorber suspension (vol. ads. suspension), heparin plasma (plasma), and the endotoxin solution [50 ng/ml] (ET solution) were pipetted into test tubes according to the specifications of Table 5. As controls, a test tube without adsorber (control without ads.) and a test tube having untreated heparin plasma (native plasma) were carried along.

TABLE 5
Test batches

| Adsorber | Vol. Ads. suspension | | Plasma | ET solution |
|---|---|---|---|---|
| # 1822 | 600 | μL | 2100 μL | 300 μL |
| # 1822 + PMB | 600 | μL | 2100 μL | 300 μL |
| # 1823 | 600 | μL | 2100 μL | 300 μL |
| # 1823 + PMB | 600 | μL | 2100 μL | 300 μL |
| # 1824 | 600 | μL | 2100 μL | 300 μL |
| # 1824 + PMB | 600 | μL | 2100 μL | 300 μL |
| Control without ads. | 100 | μL NaCl | 800 μL | 100 μL |

| | | | |
|---|---|---|---|
| Native plasma | 0 | 100 μL | 0 |

The test batches were incubated at 37° C. for 60 minutes in the Enviro-Genie shaker.

3.4. Sampling and materials: corresponding to Sections 2.5 and 2.6.

### 3.5. Results:

The results are listed in Table 6 and shown in FIG. 2, the curves shown in FIG. 2 representing the endotoxin (ET) adsorption (in endotoxin units (EUI/ml) of PMB-coated adsorbers (mean pore size 15-20 or 30-40 nm) in the time curve.

TABLE 6
Results of batch test - endotoxin adsorption

| EU/ml | 0 min | 5 min | 15 min | 60 min |
|---|---|---|---|---|
| # 1822 | 9.37 | 6.06 | 5.46 | 4.34 |
| # 1822 + PMB | 9.37 | 2.34 | 0.18 | 0.53 |
| # 1823 | 9.37 | 4.71 | 4.84 | 3.33 |
| # 1823 + PMB | 9.37 | 2.65 | 2.16 | 0.77 |
| # 1824 | 9.37 | 5.47 | 4.30 | 3.41 |
| # 1824 + PMB | 9.37 | 0.68 | 0.36 | 0.14 |
| Control without ads. | 9.37 | 6.33 | 4.95 | 3.45 |
| Native plasma | 0 | 0 | 0 | 0 |

### 4. EXAMPLE 4

### Endotoxin Adsorption—Batch Test at Various Adsorber Final Concentrations in Heparin Plasma

Polymyxin B-coated adsorbers #1823+PMB (mean pore size 15-20 nm) and #1826+PMB (mean pore size 80-100 nm) were compared to corresponding uncoated, untreated adsorbers #1823 and #1826 with respect to the endotoxin binding at various adsorber final concentrations, namely 10%, 4%, 2%, and 1%.

### 4.1. Adsorbers and Adsorber Preparation:

The following adsorbers were prepared according to the protocol of Example 1:

1) #1823

2) #1826

3) #1823+PMB (PMB-coated)

4) #1826+PMB (PMB-coated)

The polymyxin B-coated adsorbers and the uncoated adsorbers were washed—as described in 1.2—5 times using pyrogen-free NaCl. A 50% adsorber suspension was finally produced in pyrogen-free NaCl.

4.2. Heparin plasma and endotoxin solution: corresponding to Sections 2.2 and 2.3.

### 4.3. Test Batches:

In the batch of the test, a further dilution of the endotoxin solution was performed to a final concentration of 5 ng/ml.

A total of four test batches A, B, C, and D of different adsorber final concentrations were produced, a double batch being produced for each polymyxin B-coated adsorber. For the test batches, 50% adsorber suspension (vol. ads. suspension), heparin plasma (plasma), and endotoxin solution (ET solution) were pipetted into test tubes according to the specifications of Tables 7-10. As controls, a test tube without adsorber (control without ads.) and a test tube with untreated heparin plasma (plasma) were carried along.

TABLE 7
Test batch A - Endotoxin concentration 5
ng/ml, adsorber final concentration 10%

| Adsorber | Vol. Ads. suspension | Plasma | ET solution |
|---|---|---|---|
| # 1823 | 660 μL | 2367 μL | 300 μL |
| # 1823 + PMB | 660 μL | 2367 μL | 300 μL |
| # 1826 | 660 μL | 2367 μL | 300 μL |
| # 1826 + PMB | 660 μL | 2367 μL | 300 μL |
| Control without ads. | 0 | 900 μL | 100 μL |
| Plasma | 0 | 100 μL | 0 |

TABLE 8
Test batch B - Endotoxin concentration 5
ng/ml, adsorber final concentration 4%

| Adsorber | Vol. Ads. suspension | Plasma | ET solution |
|---|---|---|---|
| #1823 | 250 μL | 2575 μL | 300 μL |
| # 1823 + PMB | 250 μL | 2575 μL | 300 μL |
| # 1826 | 250 μL | 2575 μL | 300 μL |
| # 1826 + PMB | 250 μL | 2575 μL | 300 μL |
| Control without ads. | 0 | 900 μL | 100 μL |
| Plasma | 0 | 100 μL | 0 |

TABLE 9
Test batch C - Endotoxin concentration 5
ng/ml, adsorber final concentration 2%

| Adsorber | Vol. Ads. suspension | Plasma | ET solution |
|---|---|---|---|
| #1823 | 122 μL | 2639 μL | 300 μL |
| # 1823 + PMB | 122 μL | 2639 μL | 300 μL |
| # 1826 | 122 μL | 2639 μL | 300 μL |
| # 1826 + PMB | 122 μL | 2639 μL | 300 μL |
| Control without ads. | 0 | 900 μL | 100 μL |
| Plasma | 0 | 100 μL | 0 |

TABLE 10
Test batch D - Endotoxin concentration 5
ng/ml, adsorber final concentration 1%

| Adsorber | Vol. Ads. suspension | Plasma | ET solution |
|---|---|---|---|
| # 1823 | 60 μL | 2670 μL | 300 μL |
| # 1823 + PMB | 60 μL | 2670 μL | 300 μL |
| # 1826 | 60 μL | 2670 μL | 300 μL |
| # 1826 + PMB | 60 μL | 2670 μL | 300 μL |
| Control without ads. | 0 | 900 μL | 100 μL |
| Plasma | 0 | 100 μL | 0 |

The test batches were incubated at 37° C. for 60 minutes in the Enviro-Genie shaker.

### 4.4. Sampling:

All steps of the batch test and the LAL test were performed in glass LAL test tubes (T100, with plastic cap) from Chromogenix (also dilutions and standard curves). Only the sampling for centrifuging was performed in pyrogen-free microcentrifuge tubes. After 5, 15, and 60 minutes incubation time, the tubes were vortexed. A 0.3 mL sample was taken in each case and transferred into pyrogen-free microcentrifuge tubes. The samples were centrifuged in a centrifuge (Eppifuge) for 10 minutes at 11,000 g. 50 μL supernatant was withdrawn, transferred into glass LAL test tubes with 450 μL LAL water, immediately incubated at 75° C. for 5 minutes, applied to a microtitration plate, and tested in the LAL test.

4.5. Materials: corresponding to sections 2.5 and 2.6.

### 4.6. Results:

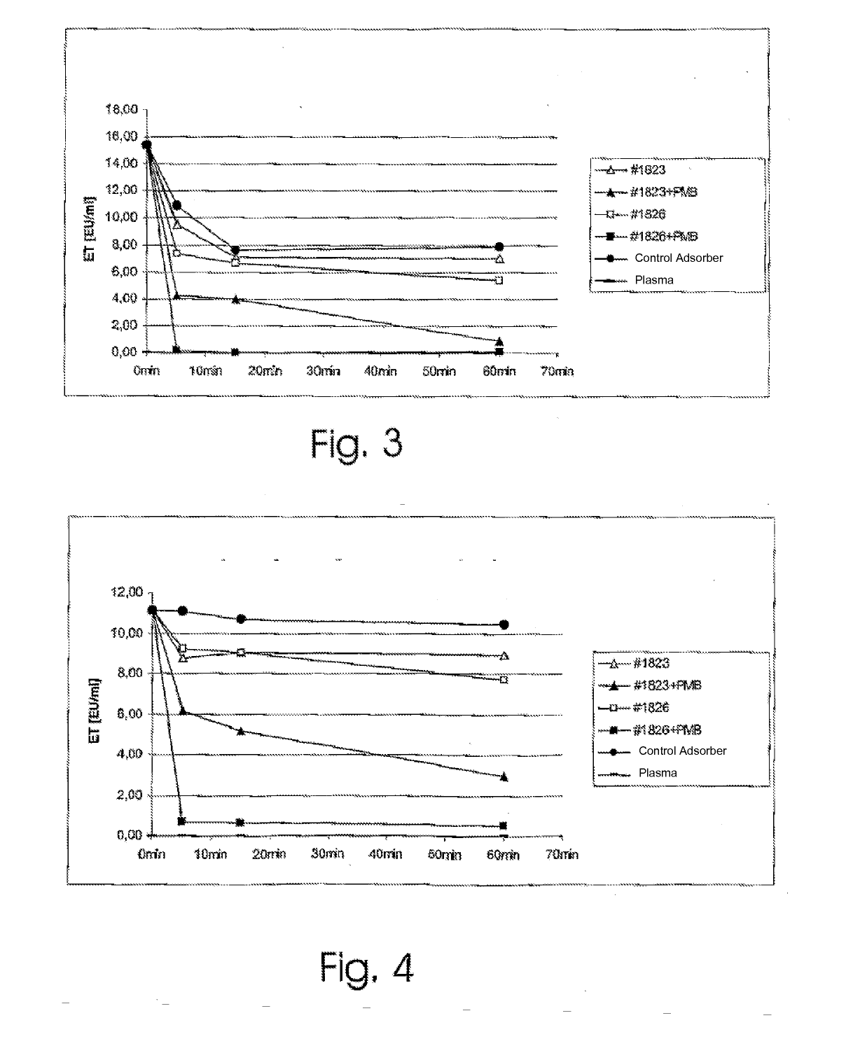

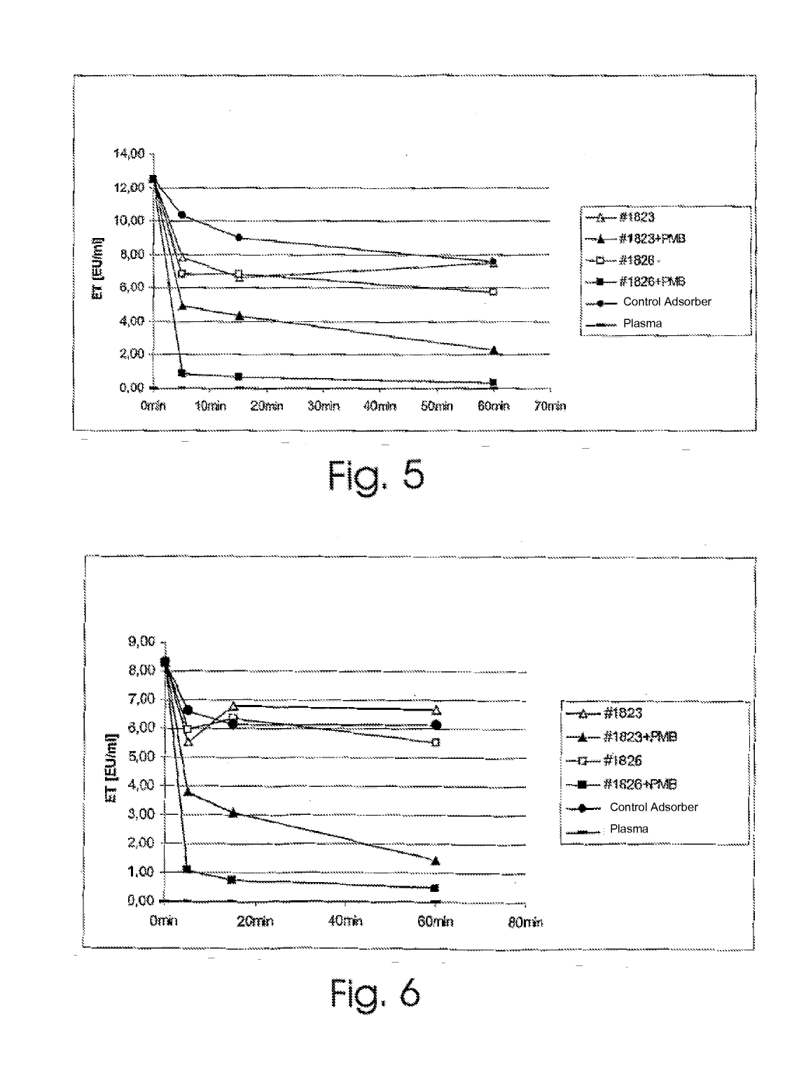

The results are listed in Tables 11, 12, 13, and 14 and shown in FIG. 3, FIG. 4, FIG. 5, and FIG. 6. The endotoxin (ET) adsorption (in endotoxin units (EU)/ml) is shown in the time curve. The tables and figures show:

TABLE 11
Table 11 and FIG. 3: Results of the batch test (test
batch A) with a 10% adsorber concentration:

| EU/ml | 0 min | 5 min | 15 min | 60 min |
|---|---|---|---|---|
| # 1823 | 15.39 | 9.55 | 7.07 | 7.03 |
| # 1823 + PMB | 15.39 | 4.28 | 4.00 | 0.90 |
| # 1826 | 15.39 | 7.33 | 6.70 | 5.37 |
| # 1826 + PMB | 15.39 | 0.19 | 0 | 0 |
| Control without ads. | 15.39 | 10.91 | 7.57 | 7.91 |
| Native plasma | 0 | x | x | 0 |

TABLE 12
Table 12 and FIG. 4: Results of the batch test (test
batch B) with a 4% adsorber concentration:

| EU/ml | 0 min | 5 min | 15 min | 60 min |
|---|---|---|---|---|
| # 1823 | 11.12 | 8.74 | 9.03 | 8.94 |
| # 1823 + PMB | 11.12 | 6.16 | 5.14 | 2.96 |
| # 1826 | 11.12 | 9.21 | 9.02 | 7.70 |
| # 1826 + PMB | 11.12 | 0.69 | 0.62 | 0.35 |
| Control without ads. | 11.12 | 11.10 | 10.69 | 10.45 |
| Native plasma | 0 | x | x | 0 |

TABLE 13
Table 13 and FIG. 5: Results of the batch test (test
batch C) with a 2% adsorber concentration:

| EU/ml | 0 min | 5 min | 15 min | 60 min |
|---|---|---|---|---|
| # 1823 | 12.49 | 7.85 | 6.66 | 7.48 |
| # 1823 + PMB | 12.49 | 4.93 | 4.33 | 2.33 |
| # 1826 | 12.49 | 6.83 | 6.82 | 5.77 |
| # 1826 + PMB | 12.49 | 0.91 | 0.65 | 0.39 |
| Control without ads. | 12.49 | 10.35 | 9.00 | 7.59 |
| Native plasma | 0 | x | x | 0 |

TABLE 14
Table 14 and FIG. 6: Results of the batch test (test
batch D) with a 1% adsorber concentration:

| EU/mL | 0 min | 5 min | 15 min | 60 min |
|---|---|---|---|---|
| # 1823 | 8.29 | 5.54 | 6.80 | 6.65 |
| # 1823 + PMB | 8.29 | 3.78 | 3.09 | 1.43 |
| # 1826 | 8.29 | 5.95 | 6.33 | 5.54 |
| # 1826 + PMB | 8.29 | 1.07 | 0.75 | 0.47 |
| Control without ads. | 8.29 | 6.61 | 6.11 | 6.11 |
| Native plasma | 0 | x | x | 0 |

### 5. EXAMPLE 5

### Endotoxin Adsorption Batch Test at an Endotoxin Concentration of 1 ng/ml.

Polymyxin B-coated adsorbers (see Example 1) #1823+PMB (mean pore size 15-20 nm), #1824+PMB (mean pore size 30-40 nm), and #1826+PMB (mean pore size 80-100 nm) were compared to corresponding uncoated adsorbers #1823, #1824, and #1826 with respect to the endotoxin binding, the endotoxin concentration in the batch test being 1 ng/ml.

### 5.1. Adsorbers and Adsorber Preparation:

The following adsorbers were prepared according to the protocol of Example 1:

1) #1823

2) #1824

3) #1826

4) #1823+PMB (PMB-coated)

5) #1824+PMB (PMB-coated)

6) #1826+PMB (PMB-coated)

The polymyxin B-coated adsorbers and the uncoated adsorbers were washed—as described in 1.2—5 times using pyrogen-free NaCl. A 50% adsorber suspension was finally produced in pyrogen-free NaCl.

5.2. Heparin plasma corresponding to section 2.2.

### 5.3. Endotoxin Solution:

LPS Pseudomonas aeruginosa (Sigma, L7018, batch 109H4043). The portioned (each 100 μl 10⁻³ g/ml (1 mg/ml)) endotoxins, which were stored at −70° C. in microcentrifuge tubes, were thawed and admixed with 900 μL LAL water. The endotoxins were subsequently diluted further in NaCl to the required concentration of 10 ng/ml (=endotoxin solution; ET solution).

### 5.4. Test Batches:

In the batch of the test, a further dilution of the endotoxin solution was performed to a final concentration of 1 ng/ml. The adsorber final concentration was 1%. For the test batches, 50% adsorber suspension (ads. susp. 50%), heparin plasma (plasma), and the endotoxin solution [10 ng/ml] (ET solution) were pipetted into test tubes according to the specifications of Table 15. As controls, a test tube without adsorber (control without ads.) and a test tube having untreated heparin plasma (native plasma) were carried along.

TABLE 15
Test batches

| | Ads. Susp. 50% | Plasma | ET solution |
|---|---|---|---|
| # 1823 | 60 μl | 2670 μl | 300 μl |
| # 1824 | 60 μl | 2670 μl | 300 μl |
| # 1826 | 60 μl | 2670 μl | 300 μl |
| # 1823 + PMB | 60 μl | 2670 μl | 300 μl |
| # 1824 + PMB | 60 μl | 2670 μl | 300 μl |
| # 1826 + PMB | 60 μl | 2670 μl | 300 μl |
| Control without ads. | | 900 μl | 100 μl |
| Native plasma | 0 | 100 μl | 0 |

The test batches were incubated at 37° C. for 60 minutes in the Enviro-Genie shaker.

5.5. Sampling and materials: corresponding to Sections 2.5. and 2.6.

### 5.6. Results:

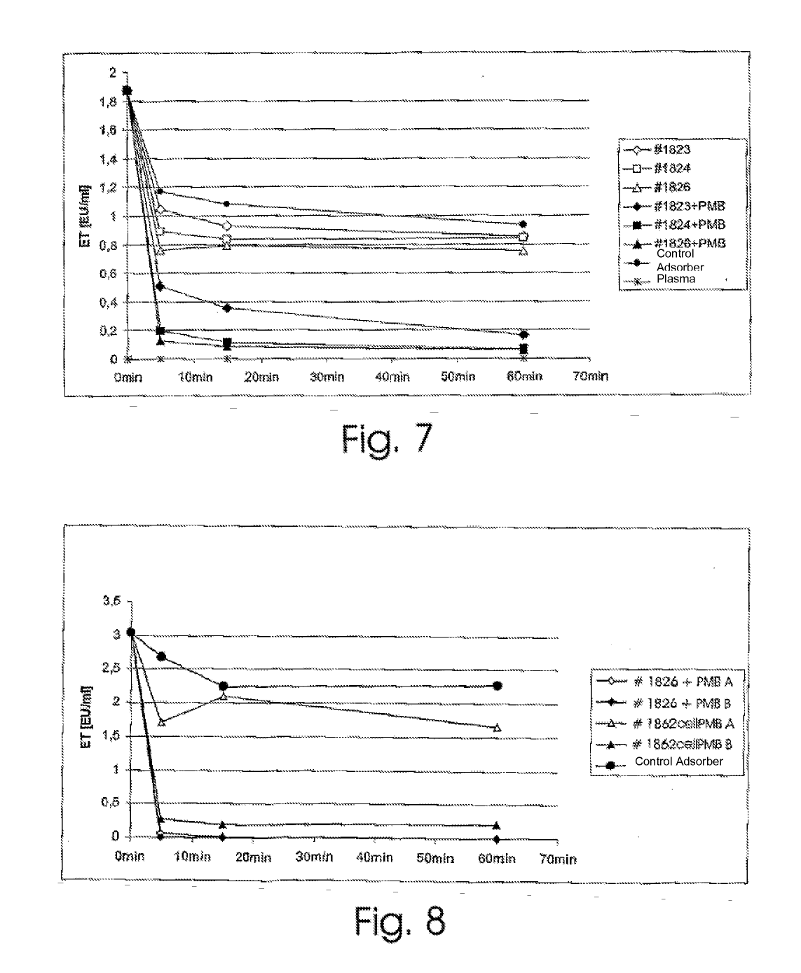

The results are listed in Table 16 and shown in FIG. 7, FIG. 7 showing a graph of the endotoxin (ET) adsorption (in endotoxin units (EU/ml) of PMB-coated adsorbers at an adsorber concentration of 1% and an endotoxin concentration of 1 ng/mL in the time curve.

TABLE 16
Results

| | 0 min | 5 min | 15 min | 60 min |
|---|---|---|---|---|
| # 1823 | 1.874 | 1.043 | 0.93 | 0.85 |
| # 1824 | 1.874 | 0.89 | 0.83 | 0.84 |
| # 1826 | 1.874 | 0.758 | 0.79 | 0.75 |
| # 1823 + PMB | 1.874 | 0.509 | 0.35 | 0.16 |
| # 1824 + PMB | 1.874 | 0.195 | 0.115 | 0.067 |
| # 1826 + PMB | 1.874 | 0.128 | 0.089 | 0.062 |
| Control without ads. | 1.874 | 1.169 | 1.08 | 0.93 |
| Native plasma | x | x | x | 0 |

### 6. EXAMPLE 6

### Testing of the Autoclaving Capability of Polymixin B-Coated Adsorbers

The autoclaving capability of a polymyxin B-coated adsorber #1826+PMB (mean pore size 80-100 nm) was compared to an adsorber based on cellulose, in which polymyxin B is covalently bound to the cellulose. The influence of the autoclaving on the endotoxin binding was studied.

### 6.1. Adsorbers and Adsorber Preparation

Two 50% adsorber suspensions (A and B) of a polymyxin B-coated adsorber #1826+PMB were produced according to the protocol of Example 1.

In addition, two 50% adsorber suspensions (A and B) of a particulate cellulose adsorber #1862 Cell+PMB, which is modified with polymyxin B, were produced (cellulose particles Fischer, lot: EA 13/1 PCKT 038, activated using epichlorohydrin and reacted with polymyxin B=>covalent bonding of the polymyxin B molecules to the cellulose particles).

The respective adsorber suspensions A, #1826+PMB A and #1862 Cell+PMB A were autoclaved at 121° C. for 20 minutes. The respective adsorber suspensions B, #1826+PMB B and #1862 Cell+PMB B, were not autoclaved:

1) #1826+PMB A (autoclaved)

2) #1826+PMB B (not autoclaved)

3) #1862 Cell+PMB A (autoclaved)

4) #1862 Cell+PMB B (not autoclaved)

600 μL of each of the 50% adsorber suspensions was placed in the provided heparin plasma in LAL test tubes (Ch. River Endosafe, pyrogen-free) T100.

6.2. Heparin plasma: corresponding to section 2.2.

### 6.3. Endotoxin Solution:

LPS Pseudomonas aeruginosa (Sigma, L7018, batch 109H4043). The portioned (each 100 μl 10⁻³ g/ml (1 mg/ml)) endotoxins, which were stored at −70° C. in microcentrifuge tubes, were thawed and admixed with 900 μL LAL water. The endotoxins were subsequently diluted further in NaCl to the required concentration of 10 ng/ml (=endotoxin solution; ET solution).

### 6.4. Test Batches:

In the batch of the test, a further dilution of the endotoxin solution was performed to a final concentration of 1 ng/ml. The adsorber final concentration in the test batches was 1%. For the test batches, 50% adsorber suspension (vol. ads. suspension), heparin plasma (plasma), and the endotoxin solution [10 ng/ml] (ET solution) were pipetted into test tubes according to the specifications of Table 17. As controls, a test tube without adsorber (control without ads.) and a test tube with untreated heparin plasma (native plasma) were carried along.

TABLE 17
Test batches

| Adsorber | Vol. Ads. suspension | Plasma | ET-Lösung |
|---|---|---|---|
| # 1826 + PMB A | 60 μL | 2670 μL | 300 μL |
| # 1826 + PMB B | 60 μL | 2670 μL | 300 μL |
| # 1862 Cell + PMB A | 60 μL | 2670 μL | 300 μL |
| # 1862 Cell + PMB B | 60 μL | 2670 μL | 300 μL |
| Control without ads. | 0 | 900 μL | 100 μL |
| Native plasma | 0 | 100 μL | 0 |

The test batches were incubated at 37° C. for 60 minutes in the Enviro-Genie shaker.

### 6.5. Sampling:

All steps of the batch test and the LAL test were performed in glass LAL test tubes (T100, with plastic cap) from Chromogenix (also dilutions and standard curves).

Only the sampling for centrifuging was performed in pyrogen-free microcentrifuge tubes.

After 5, 15, and 60 minutes incubation time, the tubes were vortexed. A 0.2 mL sample was taken in each case and transferred into pyrogen-free microcentrifuge tubes. The samples were centrifuged in a table centrifuge (Eppifuge) for 10 minutes at 11,000 g. 100 μL supernatant was transferred into glass LAL test tubes and tested in the LAL test (LAL test: Charles River Endosafe Endochrome, kit.lot: Y 1892 EK1).

### 6.6. Results:

The results are listed in Table 18 and shown in FIG. 8, FIG. 8 showing a graph of the endotoxin (ET) adsorption (in endotoxin units (EU/ml) of the autoclaved and the non-autoclaved adsorbers or the cellulose adsorbers and the controls in the time curve.

TABLE 18
Results

| EU/ml | 0 min | 5 min | 15 min | 60 min |
|---|---|---|---|---|
| # 1826 + PMB A | 3.047 | 0.062 | 0.00 | 0.00 |
| # 1826 + PMB B | 3.047 | 0.00 | 0.00 | 0.00 |
| # 1862 Cell + PMB A | 3.047 | 1.696 | 2.10 | 1.65 |
| # 1862 Cell + PMB B | 3.047 | 0.273 | 0.20 | 0.21 |
| Control without ads. | 3.047 | 2.684 | 2.24 | 2.27 |
| Native plasma | x | x | x | 0 |

The adsorbers #1826+PMB A and #1826+PMB B according to the invention had good endotoxin adsorption both before and also after the autoclaving.

In contrast, the autoclaving of the cellulose adsorber caused significant worsening of the endotoxin adsorption.

1. A sorption agent for removing endotoxins from a biological fluid, wherein:
the sorption agent includes a water-insoluble, porous carrier and polymyxin, which is immobilized on the carrier; and
the carrier has a neutral, hydrophobic surface and polymyxin is immobilized on the surface of the carrier via hydrophobic interaction.

2. The sorption agent according to claim 1, characterized in that the carrier is a hydrophobic polymer.

3. The sorption agent according to claim 2, characterized in that the hydrophobic polymer is a cross-linked polystyrene polymer, in particular a polystyrene-divinyl benzene copolymer.

4. The sorption agent according to claim 1, characterized in that the carrier has a mean pore size of at least 15 nm, preferably at least 30 nm.

5. The sorption agent according to claim 1, characterized in that the carrier has a mean pore size of not greater than 120 nm.

6. The sorption agent according to laim 4, characterized in that the carrier has a mean pore size of approximately 80 -100 nm.

7. The sorption agent according to claim 1, characterized in that the sorption agent has the form of microparticles.

8. The sorption agent according to claim 7, characterized in that the microparticles have a particle size of 20 μm or less, preferably a particle size of 8 μm or less, ideally 5 μm or less.

9. A sorption apparatus, containing a sorption agent wherein:
the sorption apparatus is configured so that the sorption agent removes endotoxins from a biological fluid;
the sorption agent includes a water-insoluble, porous carrier and polymyxin, which is immobilized on the carrier; and
the carrier has a neutral, hydrophobic surface and polymyxin is immobilized on the surface of the carrier via hydrophobic interaction.

10. A plasma circuit, containing a suspension of a sorption agent, wherein:
the sorption agent includes a water-insoluble, porous carrier and polymyxin, which is immobilized on the carrier; and
the carrier has a neutral, hydrophobic surface and polymyxin is immobilized on the surface of the carrier via hydrophobic interaction.

11. A method for removing endotoxins from a biological fluid, characterized in that a biological fluid contaminated with endotoxins is brought into contact with a sorption agent, wherein:
the sorption agent includes a water-insoluble, porous carrier and polymyxin, which is immobilized on the carrier; and
the carrier has a neutral, hydrophobic surface and polymyxin is immobilized on the surface of the carrier via hydrophobic interaction.

12. The method according to claim 11, characterized in that the biological fluid is blood or blood plasma.

13. The sorption agent according to laim 1 for the treatment of a sepsis.

14. The sorption apparatus according to claim 9 for the treatment of a sepsis.

15. The plasma circuit according to claim 10 for the treatment of a sepsis.

16. A Microspheres-based Detoxification System (MDS) configured to use a sorption agent, wherein:
the sorption agent comprising a water-insoluble, porous carrier and polymyxin, which is immobilized on the carrier; and
the carrier has a neutral, hydrophobic surface and polymyxin is immobilized on the surface of the carrier via hydrophobic interaction.
